(19) United States
(12) Patent Application Publication
(10) Pub. No.: US 2015/0208930 A1
(43) Pub. Date: Jul. 30, 2015

(54) METHOD AND MEDICAL IMAGING FACILITY FOR DETERMINING PERFUSION

(71) Applicant: Siemens Aktiengesellschaft, Muenchen (DE)

(72) Inventor: Peter Gall, Buckenhof (DE)

(73) Assignee: SIEMENS AKTIENGESELLSCHAFT, Muenchen (DE)

(21) Appl. No.: 14/607,165

(22) Filed: Jan. 28, 2015

(30) Foreign Application Priority Data
Jan. 28, 2014 (DE) 102014201499.9

(51) Int. Cl. A61B 5/026; A61B 5/0275; A61B 5/00; A61B 5/055

(52) U.S. Cl. CPC A61B 5/0263; A61B 5/055; A61B 5/0275; A61B 5/742; A61B 2576/00

(57) ABSTRACT

In a method and apparatus for determining perfusion within a tissue region of the human or animal body, the tissue region being supplied by at least two input vessels, a time series of perfusion measurements is acquired, a first concentration/time curve at a first input vessel is determined, a second concentration/time curve at a second input vessel is determined, and a third concentration/time curve in the tissue region is determined. A weighting factor of the supply to the tissue region by the first input vessel and/or the second input vessel is determined by maximizing a residue function that is a function of the weighting factor.

### BACKGROUND OF THE INVENTION

1. Field of the Invention

The invention relates to a method and a medical imaging facility for determining perfusion within a tissue region of a human or animal body.

2. Description of the Prior Art

Perfusion measurements on tissue, namely measurements of the blood through-flow state in the tissue, are known in principle and supply valuable information, for example in relation to strokes which can result in a partial reduction of brain perfusion, or the blood through-flow in the prostate (T1-weighted perfusion) in the case of magnetic resonance imaging.

In known methods, a contrast agent is injected into the patient in the form of a bolus and its inflow and outflow through the capillaries of the tissue is observed using medical imaging methods. The contrast agent here is generally injected into a vein. The contrast agent is detected for example using tools provided by magnetic resonance tomography (MRT) or by x-ray imaging.

The physiology of the body and dispersion effects, however, cause contrast agent injected into a vein, for example, to not reach the tissue region under examination at a fixed time point after injection nor does it follow the concentration/time predetermined by injection. The signal measured in the tissue region, in other words the propagation of the contrast agent there, therefore thus provides little information by itself and is not suitable for a comparative evaluation over time.

In order to obtain perfusion data for the tissue region that can be evaluated with respect to some type of baseline, in known methods the tissue signal is compared with the associated blood supply. This comparison is then used to calculate a theoretical tissue signal, which can then be evaluated with regard to the tissue signal obtained in an earlier examination, so as to identify changes therein over time.

In the prior art the procedure is as follows:

The propagation of the contrast agent over time is measured both in the tissue region of interest and in the vessel supplying the tissue region with blood, for example using MR perfusion measurement. Here the supply from the vessel is assumed for example from averages over a population. The contrast agent concentration as a function of time in the vessel supplying the tissue region (generally an artery) is generally referred to here as the arterial input function (AIF).

When the data have been recorded, the concentration curve (contrast agent concentration as a function of time) measured in the tissue is mathematically deconvoluted with the arterial input function (AIF). This gives a so-called residue function, which describes a theoretical and comparable propagation of the contrast agent in the corresponding tissue region. The following relationship applies:

c(t)=Flow*c_{Artery}(t)×R(t)

wherein c(t) is the time-dependent tissue concentration, Flow is the flow by way of the supplying vessel, c_{Artery}(t) is the time-dependent concentration in the supplying vessel (Artery), R(t) is the time-dependent residue function and x is the mathematical convolution operator.

The residue function corresponds to a concentration/time curve, which would result in the tissue in response to an ideal, ultra-short contrast agent bolus (contrast agent impulse, impulse function) and can therefore also be referred to as the tissue impulse response function. It therefore describes the blood through-flow state (perfusion) in the tissue region under the theoretical assumption of an ideal contrast agent impulse. The residue function can be determined for example for each individual voxel or for defined segments of the tissue region of interest.

For some tissue regions in human and animal bodies, the problem arises with the above procedure that the tissue regions are not supplied by a single supplying vessel, but by two or more vessels. One example of such tissue with two “inputs” is the liver. Liver tissue is supplied by an artery and a portal vein (vena portae). For a precise understanding of the blood through-flow in such tissue, both or all the “inputs” (all the vessels supplying the tissue) must be known. For the most precise determination possible of the residue function of such tissue, it is desirable to know the corresponding weighting of the individual “inputs” (proportion of the overall supply to the tissue). The relative weighting factors can also provide further valuable information. For example healthy liver tissue has different weighting factors from tumor tissue. Knowledge of the weighting factors therefore has a diagnostic value beyond the precise determination of the residue function.

Such solutions generally use modifications of the so-called Tofts model with two inputs (for example KOH, Radiology, Vol. 249, 2008). In this process additional parameters are introduced into the basic Tofts model (Tofts, MRM, Vol. 17, 1991).

### SUMMARY OF THE INVENTION

An object of the invention is to provide a method and an apparatus for determining perfusion within a tissue region, which allow the most precise determination possible of perfusion when there are a number of vessel inputs to the tissue region. The method should in particular be as robust and/or fast as possible and/or should allow model-independent determination of perfusion.

According to the invention, such a method includes the steps of acquisition of a time series of perfusion measurements, determination of a first signal/time curve at a first input vessel, determination of a second signal/time curve at a second input vessel, and determination of a third signal/time curve in the tissue region, and determination of a weighting factor of the supply to the tissue region by the first input vessel and/or the second input vessel, by maximizing a residue function that is a function of the weighting factor and making the weighting factor available at an output of the computer as an electronic signal.

The residue function describes perfusion, in other words the blood through-flow state, in particular the quantity of contrast agent present in the tissue at certain time points, preferably for each voxel of the tissue region of interest, assuming an ideal bolus in the form of an impulse, as described above.

The signal/time curves can be in particular intensity/time curves or concentration/time curves. A signal/time curve can therefore be understood in particular as the contrast agent concentration as a function of time or a concentration/time curve. With MRT the measurable signal drop is approximately proportional to the contrast agent concentration. The measured signal is therefore preferably inverted here in order to produce a signal/time curve for the inventive method.

According to the invention, maximization of the residue function is understood in particular as maximization of a highest value (peak) of the residue function. It refers in particular to the selection or definition of the residue function, the highest value (peak) of which has the greatest value. In some embodiments the amount of the highest apex point is maximized. According to the invention the residue function is determined in particular taking into account the at least two input vessels and the weighting factors underlying said residue function are determined for the input vessels.

When there are two vessel inputs for a tissue region, the following relationship exists between the measured tissue concentration (concentration/time curve of the tissue region) and the concentrations in the input vessels (concentration/time curves of the input vessels):

c(t)=Flow*(a*c1(t)+(1−a)*c2(t))×R(t) (1),

wherein c(t) is the time-dependent tissue concentration (tissue curve), Flow is the flow, in particular the overall flow, by way of the supplying vessels, a is the relative weighting factor of a first input vessel, c1(t) is the time-dependent concentration in the first input vessel, c2(t) is the time-dependent concentration in a second input vessel, R(t) is the time-dependent residue function and x is the mathematical convolution operator.

The residue function R therefore relates, as described above, to the tissue impulse response function and can be determined by a mathematical deconvolution of the equation (1) for a given value of a (0<a<1). It is therefore a function of the weighting factor a.

If the input or inputs described by the signal/time curve in the tissue region is/are not correctly described due to an inappropriate value of a, this error is compensated for mathematically by the residue function R. According to the invention it has been identified here that such compensation is manifested in a widening of the residue function R. As the total quantity of blood passing by way of the individual input vessels into the tissue region is identical for any value of a, the widening of the residue function R also brings about a lowering of its highest value (peak, maximum value, upper or lower apex point).

According to the invention, the weighting factor, or where applicable the weighting factors, should be determined so that the residue function R assumes a maximum highest value, and therefore the smallest width. The optimum value for the weighting factor in accordance with the invention is therefore the value that produces the highest peak in the residue function R. Maximization of the residue function, in particular maximization of the preferably first peak of the residue function, can be performed analytically or numerically. With the numerical solution the residue function is calculated repeatedly for different weighting factors. The solution containing the highest peak or greatest maximum value is then selected from the number of solutions.

In a preferred embodiment of the invention, the residue function is determined by deconvolution of the sum of the weighted signal/time curves (concentration/time curves). Deconvolution (mathematical deconvolution) is a known method and thus need not be described in greater detail herein.

A value at which the residue function that is a function of the weighting factor reaches a maximum apex value is preferably determined as the value for the weighting factor. The weighting factor is therefore defined in such a manner that with this weighting factor the time-dependent residue function has an apex value, in particular a maximum concentration, which is greater in amount than the apex values of the other solutions. In particular only the first maximum value of the residue function, which relates to a first passage of the contrast agent, is taken into account here. Further maxima, which relate to subsequent passages, are generally smaller and are not taken into account.

In another preferred embodiment of the invention a specific weighting factor is selected for each considered input vessel of the tissue region of interest. The weighting factors for the individual input vessels are preferably selected in such a manner here that the sum of the weighting factors corresponds to a predetermined value, preferably the value 1. The sum of the weighting factors for all the considered input vessels is therefore constant and is preferably 1. This also applies to each considered voxel of the tissue region.

To determine perfusion within the tissue region the respective input vessels are preferably first determined or defined for the relevant tissue region. This can be done for example using an angiography image, in particular with computer assistance. The image used to determine the input vessels is preferably an anatomical image recorded before the acquisition of the time series, preferably already containing contrast agent. It can also be an image from the time series. The angiography image can be recorded in particular using the subtraction angiography (DSA) method, which is known in principle. The image here only contains the filled vessels, without background, for example bones.

In a further preferred embodiment of the invention the weighting factor and/or the residue function is represented graphically on a display facility. The invention therefore also relates to a method for representing the perfusion of a tissue region, perfusion being represented by representing at least one weighting factor and/or representing the residue function. To this end the residue function or the at least one weighting factor is first determined according to the invention and then represented on the display facility, for example a monitor. The representation of the residue function and/or of the weighting factor or weighting factors can be used to determine further parameters of the blood through-flow, for example the blood volume or the blood flow.

In another particularly preferred embodiment the weighting factor and/or residue function is determined and/or represented with spatial resolution for a plurality of segments of the tissue region. The weighting factor and/or residue function can in particular be determined and/or represented pixel by pixel or in three-dimensional space (voxel by voxel).

The perfusion measurement, in particular the acquisition of the time series of perfusion measurements, preferably takes place using magnetic resonance tomography (MRT). In this process preferably one or more sectional images of the tissue region of interest including the supplying vessels (input vessels) are produced, preferably in a time series with 40-200, preferably 60-120 repetitions per minute.

According to the invention the determination of the at least one weighting factor takes place in particular independently of a model, in other words without a model or not based on a model. The weighting factor and/or residue function is preferably, in particular exclusively, determined on the basis of MR perfusion data for the tissue and the supplying vessels.

The inventive medical imaging apparatus for determining perfusion within a tissue region of the human or animal body, which is supplied by way of at least two input vessels, is configured in particular to perform the inventive method, and has a perfusion data acquisition unit that is operated to acquire a time series of perfusion measurements and a computer designed to determine a first signal/time curve at a first input vessel, a second signal/time curve at a second input vessel and a third signal/time curve in the tissue region, and the computer is also designed to determine a weighting factor for the supply to the tissue region by way of the first input vessel and/or the second input vessel by maximizing a residue function that is a function of the weighting factor, and to make this weighting factor available as an electronic signal at an output of the computer.

The advantages and effects described in relation to the method are achieved with the apparatus. The imaging facility according to the invention in particular allows the perfusion of a tissue region supplied by way of at least two input vessels to be determined and/or represented precisely.

In a preferred embodiment, a display is provided in communication with the computer at which the computer causes the at least one weighting factor and/or the residue function to be displayed graphically. The display facility can be configured in particular to represent the weighting factor or where applicable the weighting factors and/or the residue function based on a mathematical function or a graph. The residue function is preferably represented here as a function of time. The weighting factors can be represented in particular with spatial resolution.

### BRIEF DESCRIPTION OF THE DRAWINGS

FIG. 1 is a flowchart that illustrates steps of an exemplary embodiment of the inventive method.

FIG. 2 shows an exemplary embodiment of an apparatus for performing the inventive method.

FIG. 3 shows a graphic representation of the residue function for an exclusively arterial input, an input exclusively by a portal vein and a combined input by an artery and portal vein with a weighting factor of 0.1.

FIG. 4 shows a graphic representation of the residue function for an exclusively arterial input, an input exclusively by a portal vein and a combined input by an artery and portal vein with a weighting factor of 0.3 and

FIG. 5 shows a graphic representation of the residue function for an exclusively arterial input, an input exclusively by a portal vein and a combined input by an artery and portal vein with a weighting factor of 0.9.

### DESCRIPTION OF THE PREFERRED EMBODIMENTS

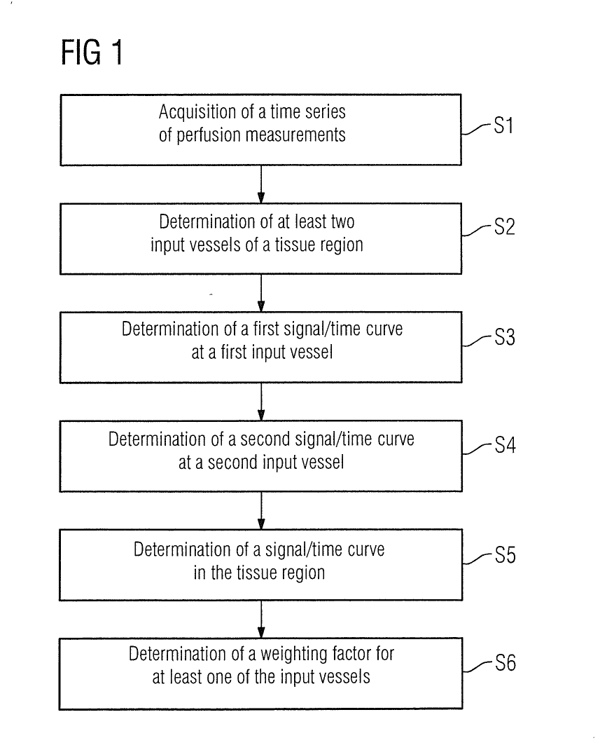

The individual method steps according to one exemplary embodiment of the inventive method are described in the following with reference to FIG. 1.

In a first step S1 a time series of perfusion measurements (perfusion data, perfusion images) is recorded, for example by means of MR perfusion imaging, in particular by means of digital subtraction angiography (DSA). Recording includes the recording of a plurality of temporally consecutive angiography images (image series) both of the tissue region of interest and of the supplying vessels. The recordings are performed after injection of a contrast agent bolus, for example with a temporal resolution of at least one image per second, preferably at least two images per second. A so-called mask image is subtracted from the image series, said mask image being recorded without contrast agent. The result is a recording sequence which represents the flow of contrast agent into the body region of interest.

Step S1 can be performed for example using an apparatus for performing a dynamic, contrast agent-assisted perfusion MRT. This allows the acquisition of preferably one or more sectional images, which contain the tissue region of interest and the supplying vessels.

In a step S2 at least two, preferably all the input vessels of the tissue region of interest to be taken into account are determined. For example a tissue region can be supplied by way of an artery and a portal vein or by way of two arteries. The relevant input vessels can be determined manually by a user or with computer assistance, for example by expert (trainable, neural network) systems, based on an angiography image.

In a step S3 a first signal/time curve at a first input vessel is determined from the recorded data record of the perfusion measurement. The signal/time curve is in particular an intensity/time curve or a concentration/time curve, in other words the progression over time of the signal, the intensity or the contrast agent concentration in the first input vessel.

In a step S4 a second signal/time curve at a second input vessel is determined from the recorded data record of the perfusion measurement. The signal/time curve is an intensity/time curve or a concentration/time curve, in other words the progression over time of the signal, of the intensity or of the contrast agent concentration in the second input vessel.

In a step S5 a signal/time curve of the tissue region is determined from the recorded data record of the perfusion measurement. The signal/time curve is in particular an intensity/time curve or a concentration/time curve, in other words the progression over time of the signal, the intensity or the contrast agent concentration in the tissue region.

In a step S6 a weighting factor for at least one of the input vessels is finally determined using the signal/time curves determined in steps S3 to S5. A further weighting factor for a second input vessel for example can then be calculated therefrom. The further weighting factor or factors can be determined in that the sum of the weighting factors corresponds to a predetermined value, for example 1.

The weighting factor and the associated residue function are determined by maximizing the number of residue functions in such a manner that the residue function having the greatest maximum value (peak) is selected.

The corresponding residue function is determined in association with the at least one determined weighting factor.

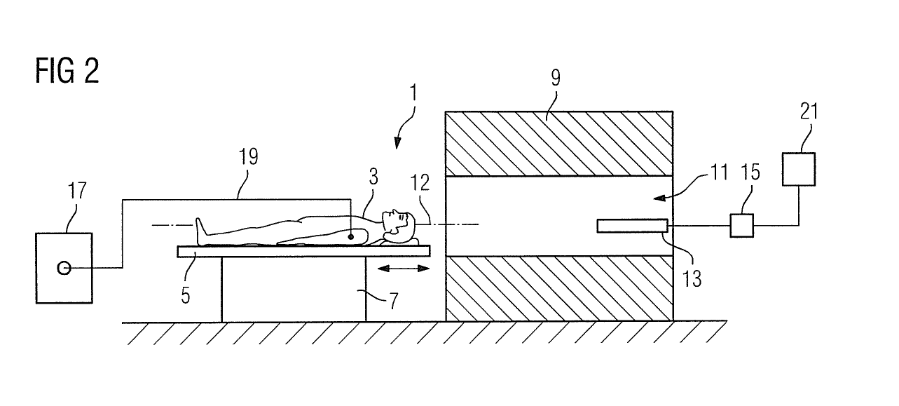

FIG. 2 shows an apparatus for performing the inventive method. The apparatus is set up to perform dynamic, contrast agent-based perfusion MRT, in which the propagation of a contrast agent in the tissue region of interest is detected. To this end the contrast agent is injected intravenously as a bolus. At the same time or shortly after injection the signal rise or signal drop caused by the contrast agent is measured.

The apparatus, which can generally be referred to as a medical imaging facility, comprises an acquisition facility 1 for acquiring a dynamic perfusion data record and is a nuclear spin tomography device in the illustrated embodiment. It serves to examine a region of an examination object or patient 3 using imaging. The patient 3 is shown in a pre-examination state, lying on a bed 5, which is held by a support 7 and can be displaced horizontally along an axis 12.

In order to produce a magnetic field the acquisition facility 1 has an in particular superconducting magnet 9, in the opening 11 of which the actual examination takes place. A transmit coil (not shown in detail) radiates high-frequency impulses into the patient 3 who has been introduced into the opening 11. Echo impulses originating from the patient 3 are recorded by a high-frequency coil arrangement 13 and fed to a computation facility 15. The computation facility 15 is configured to determine or calculate the at least one weighting factor and/or the residue function. The weighting factor and/or the residue function are displayed on a display facility 21, for example a monitor.

An injection facility 17 is provided for injecting the contrast agent. The contrast agent is injected into the patient 3 preferably automatically by way of an injection line 19.

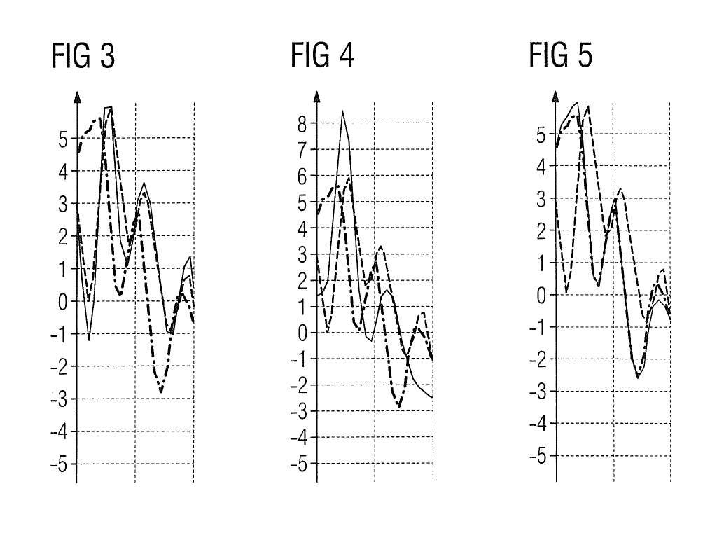

FIGS. 3 to 5 show examples of a residue function R of a tissue region, which is supplied by way of two vessel inputs (for example artery and portal vein). The residue function R assuming an exclusive supply by a first input vessel (for example artery) is shown with a broken line in each instance. The dot/dash line shows the residue function R assuming an exclusive supply by a second input vessel (for example portal vein). The continuous line shows the residue function R assuming a supply by two input vessels (both the artery and the portal vein), according to equation (1). FIG. 3 shows the residue function R for a value of a=0.1, FIG. 4 the residue function R for a value of a=0.3 and FIG. 5 the residue function R for a value of a=0.9. It can be seen that the highest peak of the residue function R is reached at a=0.3. From these values of a therefore a=0.3 is the appropriate value (the one that should be selected) for the weighting factor.

It should be noted that the further maxima of the illustrated residue function R relate to further passages of the contrast agents; generally however only the first passage (in other words the first maximum) is taken into account. Negative values of the residue function are produced by numerical resolution of the equation (1) and have no physiological relevance.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for determining perfusion within a tissue region of a living subject, said tissue region being supplied with blood by at least two input vessels, said method comprising:
operating a perfusion data acquisition unit, while a living subject is situated therein, to acquire a time series of perfusion measurements;
providing said time series to a computer and, in said computer, automatically determining a first concentration/time curve at a first of said input vessels, determining a second concentration/time curve at a second of said input vessels, and determining a third concentration/time curve in said tissue region;
in said computer, automatically determining a weighting factor of the blood supply to the tissue region via at least one of said first of said input vessels and said second of said input vessels, by maximizing a residue function that is a function of said weighting factor; and
making the determined weighting factor available at an output of said computer as an electronic signal.

2. A method as claimed in claim 1 comprising determining said residue function in said computer by deconvolution of a sum of the respective weighted concentration/time curves.

3. A method as claimed in claim 1 comprising determining, as a value for said weighting factor, a value of said weighting factor at which said residue function reaches a maximum apex value.

4. A method as claimed in claim 1 comprising selecting a weighting factor for each of said at least two input vessels, which cause a sum of the weighting factors for all of the input vessels to correspond to a predetermined value.

5. A method as claimed in claim 4 wherein said predetermined value is 1.

6. A method as claimed in claim 1 comprising determining said at least two input vessels from an angiography image.

7. A method as claimed in claim 1 comprising providing said electronic signal from said computer to a display in communication with said computer and, at said display, graphically representing at least one of said weighting factor and said residue function.

8. A method as claimed in claim 1 comprising determining at least one of said weighting factor and said residue function with a spatial resolution for a plurality of segments within said tissue region.

9. A medical imaging apparatus for determining perfusion within a tissue region of a living subject, said tissue region being supplied by at least two input vessels, said apparatus comprising:
a perfusion data acquisition unit;
a control computer configured to operate the perfusion data acquisition unit, while a living subject is situated therein, to acquire a time series of perfusion measurements;
weighting factor determination computer configured to automatically determine a first concentration/time curve at a first of said input vessels, a second concentration/time curve at a second of said input vessels, and a third concentration/time curve in said tissue region;
said weighting factor determination computer being configured to automatically determine a weighting factor of the blood supply to the tissue region via at least one of said first of said input vessels and said second of said input vessels, by maximizing a residue function that is a function of said weighting factor; and
said weighting factor determination computer being configured to make the determined weighting factor available at an output of said weighting factor determination computer as an electronic signal.

10. A medical imaging apparatus as claimed in claim 9 comprising a display in communication with said weighting factor determination computer, and wherein said weighting factor determination computer is configured to produce a graphical representation of at least one of said weighting factor and said residue function at said display.
